(19) United States
(12) Patent Application Publication
(10) Pub. No.: US 2025/0010006 A1
(43) Pub. Date: Jan. 9, 2025

(54) SELECTIVE LOBE DELIVERY OF THERAPEUTICS AND APPARATUS FOR INTERVENTIONAL BRONCHOSCOPY

(71) Applicant: Luiz Maracaja, Winston-Salem, NC (US)

(72) Inventor: Luiz Maracaja, Winston-Salem, NC (US)

(21) Appl. No.: 18/711,554

(22) PCT Filed: Nov. 17, 2022
(86) PCT No.: PCT/US2022/050330

§ 371 Date: May 17, 2024

Related U.S. Application Data
(60) Provisional application No. 63/281,012, filed on Nov. 18, 2021.

(51) Int. Cl. A61M 16/04

(52) U.S. Cl. CPC A61M 16/0404; A61M 16/045

(57) ABSTRACT

Certain embodiments are directed to the use of an airway device that allows for selective lobar isolation for the administration of drugs and biologies in liquid solution deeply into lung tissue, with simultaneous ventilation of the rest of the lungs. In addition, the novel airway device facilitates the performance of interventional bronchoscopy procedures such as biopsies, removal of foreign bodies, tumor debulking and placement of stents. The advantage of having separate ventilation and working channels allows continuous view and use of instruments without interruption necessary to remove structures from the airway.

### PRIORITY PARAGRAPH

This Application is an International Application claiming priority to U.S. Provisional Application 63/281,012 filed Nov. 18, 2021, which is incorporated herein by reference in its entirety.

### BACKGROUND

The alveolar membrane is a thin and delicate cellular layer with a large surface in contact with the atmosphere to promote gas exchange with the blood. The tracheobronchial tree and its multiple bifurcations transport air from the atmosphere to the alveoli and constitute a defense mechanism against aspiration of particulate matter, environmentally inhaled particles, and aerosols that can carry microorganisms and environmental pollutants.

Respiratory diseases have always haunted humankind, and despite significant improvement in mechanical ventilation, therapeutic arsenal, and deep understanding of disease mechanisms, the delivery of therapies in the lung parenchyma faces pharmacodynamics challenges. The tracheal-bronchial complex anatomy, high perfusion, and absorption capacity limit the therapeutic options that can be administered by the inhalation route.

The inhalation of small particles has been used for many decades as the main therapy route for multiple diseases, including COPD, asthma, pulmonary hypertension, and other lung diseases, with the advantage of rapid onset and less systemic effects. Aerosol particle deposition in the alveoli and lower respiratory tract depends on several factors such as the type of ventilation, spontaneous vs mechanical ventilation, particle size or median aerodynamic diameter (MMAD), the inspiratory flow velocity, duration of the inspiratory pause, and regional ventilation. A significant number of aerosolized therapies are deposited on the upper airway and tracheobronchial tree, where it is systemically absorbed.

In other words, the same defense filter that prevents harmful aerosol particles from reaching the alveolar level is also the main barrier that prevents the delivery of multiple therapies that would potentially benefit various pulmonary disease processes. For instance, antibiotics in lobar pneumonia, surfactant in collapsed lobes caused by ARDS, gene therapy in cystic fibrosis, chemotherapy in bronco alveolar cancer [6], drugs for tuberculosis, and aspergillus in cavitary lesions, anti-inflammatory for sarcoidosis and interstitial lung disease, and ultimately cell therapy for pathologies the cause tissue necrosis with the destruction of lung parenchyma.

The aerosol delivery is more efficient with small particle size (between 2-10 microns), spontaneous ventilation, device used for nebulization, high inspiratory flows, long inspiratory pauses, and homogenous regional ventilation. The morphology and asymmetric pattern of the tracheobronchial tree bifurcation (branching points) are responsible for the impaction of the particles on the walls, and most of the particles will only reach the 7th generation of bifurcation. There are approximately 24 generations of bifurcations until the respiratory bronchi and alveolar microenvironment is achieved. As the particles travel with the airflow through the tracheobronchial tree, the bifurcations, narrowing, and change in direction create a turbulent flow that directs the particles against the wall instead of staying in the center of the lumen. The delivery of living cells, such as stem cells for regenerative medicine, would not be possible due to large size and desiccation during the aerosolizing process.

The efficacy of delivery of aerosolized particles is significantly reduced in invasive ventilation. A study with scintigraphy, cyclotron scan, and aerosolized technetium showed that only 18% of the initial dose reaches the tracheobronchial tree. The remaining 82% are distributed on the endotracheal tube respiratory circuit (filters and circuit limbs). The majority of the particles that reach the tracheobronchial tree are deposited in the central airways. A negligible amount, if any, reaches the lung's periphery, significantly crippling the therapeutic capability of invasive ventilation.

The heterogeneity of regional ventilation is also altered by pulmonary edema from the cardiogenic or inflammatory origin, such as acute respiratory distress syndrome (ARDS). ARDS is a form of respiratory failure characterized by rapid onset of bilateral pulmonary edema, critical reductions in blood oxygen content (hypoxemia), and high potential for death. Edema and surfactant dysfunction result in alveolar collapse and reduction in compliance. Since the syndrome affects certain lung regions more than others, the condition creates a significant heterogeneity of mechanical function that interferes with regional ventilation.

The injured, edematous, and collapsed parts of the lung parenchyma have minimal or no regional ventilation, and the unaffected zones receive most of the flow and aerosol therapies.

Interestingly, liquid solutions can be very effective in reaching alveoli if properly administered. It is known from research models of ARDS, that alveolar surfactant can be extracted from the alveoli with lung lavage with saline solution. However, if a large liquid volume is given into the whole lung, it can deplete surfactant, collapse, and cause severe hypoxemia. Moreover, the studies using liquid ventilation with perfluorocarbon and CT imaging showed the alveolar distribution of liquid could be achieved using a special liquid ventilator.

Ultimately the current therapeutic option for delivery in mechanical ventilation are far from ideal, the aerosol route is likely not effective, and the liquid delivery may be too risky for a patient with poor or borderline pulmonary function.

This invention also seeks to provide a solution and improvement related to bronchoscopy, interventional bronchoscopy, navigation, and robotic assisted bronchoscopy procedures.

Interventional pulmonology is an medical subspecialty that deals specifically with minimally invasive endoscopic and percutaneous procedures for diagnosis and treatment of airway, lung and pleural diseases. Bronchoscopy is an endoscopic technique of visualizing the inside of the airways for diagnostic and therapeutic purposes. Flexible bronchoscopy allows for airway inspection, the diagnosis of airway lesions, therapeutic aspiration of airway secretions, removal of foreign bodies and transbronchial biopsy to diagnose lung disorders.

Transbronchial biopsy allows sampling of lung tissue, nodules, or other suspect lesions.

A current limitation to perform bronchoscopy procedures is related the working channel size of the scopes. Since the scopes are placed through the endotracheal tube, the size of the scope and consequentially the size of the working channel are limited by the internal diameter or the tube.

Additionally, every time a structure needs to be removed and doesn't fit in the channel of the scope the entire scope needs to be removed, losing the site visualization and interrupting the procedure.

### SUMMARY OF INVENTION

Here is describe a new apparatus and methods for selective lobe delivery of drugs and biologics, tube for interventional bronchoscopy procedures. An apparatus referenced in US 20180272089A1 (which is incorporated herein by reference in its entirety), consists in an endotracheal tube and one or more lobar tubes placed under bronchoscopy guidance. The tracheal tube has an external plastic film or sheath that allows the formation of a channel for the placement of the lobar tube(s). The lobar tube has a cuff on the distal tip, the tube is advanced to a specific lobe of interest, and upon the inflation of the cuff, the lobe is isolated from the rest of the lungs, which are simultaneously ventilated using the tracheal tube.

Certain embodiments are directed to a respiratory apparatus assembly comprising (a) an endotracheal tube device and a lobar tube. The endotracheal tube has an elongated body forming a primary lumen and an expandable secondary channel or lumen formed by plastic sheath that runs along the outside of the tube. The endotracheal tube having a proximal portion that remains external to a subject during use and a tracheal tube portion that is inserted into the trachea of a subject. The tracheal tube portion having a tracheal cuff around the tracheal portion of the tube that can be expanded to seal against the trachea. The secondary expandable channel or lumen is positioned outside the endotracheal tube and inside the tracheal cuff. In certain aspects the lobar tube can be inserted in the second expandable lumen or in the endotracheal lumen. The secondary expandable channel or lumen is formed by a sheath external to the surface of the endotracheal tube. The sheath can fully encircle the endotracheal tube or be attached to the exterior surface with the exception of the portion that expands to form the lumen. In certain aspects, the endotracheal tube can have a proximal first connector on the end of the endotracheal tube. The endotracheal tube has a second connector that is positioned external to the patient and distal to the proximal first connector. The second connector has a removable cover that when removed provides access to the secondary expandable channel or lumen. The second connector can have two entry ports. The entry ports can contain silicon valves. Each entry port can have a port cap that seals the port when not in use. The valves prevent leaking during instrumentation and the cap provides seal necessary for mechanical ventilation. The lobar tube can be used as a selective delivery device comprising an elongated body forming the lobar tube having a lumen. The lobar tube is configured to be inserted into the lumen of the endotracheal tube device forming a respiratory assembly. The lobar tube having a distal inflatable lobar cuff that when inflated isolates a lobe of the lung from the rest of the lung(s). The lobar tube has a proximal connector. The lobar tube is configured to be position in one lobe of the lung and forming an assembly with isolated lumens (endotracheal lumen and a lobar lumen). The lobar lumen is configured for access through the lobar tube for delivery of therapies and the like. The endotracheal lumen is configured for access through the endotracheal tube for ventilation of the lungs. The assembly is configured for lobe isolation and for the administration of therapies in a liquid format with simultaneous ventilation of the other lung lobes to maintain safe oxygenation.

The expandable sheath and removable cap allow for the passage of objects that would not possibly pass the internal diameter of an endotracheal tube. The tracheal cuff should be deflated for passage objects. After the object's passage, the cuff is re-inflated, and the cap is reconnected to prevent leaks during mechanical ventilation.

Certain embodiments are directed to therapeutic delivery systems for delivery into lung lobes or lung segments comprising the device described herein. The therapeutic delivered is not restricted to liquids. The therapeutic can be in a liquid, aerosol, or gaseous physical state. The placement of the lobar tubes is guided by flexible bronchoscopy or robotic-assisted navigational bronchoscopy, based on radiologic imaging modalities. The delivery system for a broad range of diagnostics or therapeutics agents including drugs, biologics, and radioactive agents. The topical placement into the tissue of the specific lung lobe will prevent systemic side effects and provide more local effects. The therapeutic delivery system can be used as a delivery platform of cell or gene therapy.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

FIG. 1 illustrates the side view of the apparatus for selective lobe delivery of therapeutics—1. Endotracheal tube, 2. Inflatable cuff, 3. Lumen of the tube, 4. respiratory connector for endotracheal tube, 5. pilot balloon for tracheal cuff inflation, 6. connector for insertion and removal of the bronchial tube, 7. cap for the connector, 8. plastic film or sheath, 9. Lobar tube, 10. cuff for the lobar tube, 11. connector for the lobar tube, 12. pilot balloon for lobar tube cuff inflation.

FIG. 2 illustrates the side view of the apparatus for selective lobe delivery of therapeutics—1. Endotracheal tube, 2. Inflatable cuff, 3. Lumen of the tube, 4. respiratory connector for endotracheal tube, 5. pilot balloon for tracheal cuff inflation, 6. connector for insertion and removal of the bronchial tube, 7. cap for the connector, 8. plastic film or sheath, 9. Lobar tube, 10. cuff for the lobar tube, 11. connector for the lobar tube, 12. pilot balloon for lobar tube cuff inflation.

FIG. 3 illustrates the front view of the lungs and respiratory apparatus. 1. Endotracheal tube, 2. Inflatable cuff, 13. ventilator, 14. respiratory circuit inspiratory limb, 15. respiratory circuit expiratory limb, 17, trachea, 18. carina, 19. right upper lobe, 20. right upper lobe bronchi, 21. right middle lobe, 22. right middle lobe bronchi, 23. right lower lobe, 24. right lower lobe bronchi, 25. left upper lobe, 26. left upper lobe bronchi, 27. left lower lobe, 28. left lower lobe bronchi.

FIG. 4 illustrates the front view of the lungs and respiratory apparatus. 1. Endotracheal tube, 2. Inflatable cuff, 9. lobar tube, 10. cuff of lobar tube, 11. connector for the lobar tube, 13. ventilator, 14. respiratory circuit inspiratory limb, 15. respiratory circuit expiratory limb, 17. trachea, 18. carina, 19. right upper lobe, 20. right upper lobe bronchi, 21. right middle lobe, 22. right middle lobe bronchi, 23. right lower lobe, 24. right lower lobe bronchi, 25. left upper lobe, 26. left upper lobe bronchi, 27. left lower lobe, 28. left lower lobe bronchi, 30. flexible bronchoscope, 31. optic component of the bronchoscope.

FIG. 5 illustrates the front view of the lungs and respiratory apparatus. 1. Endotracheal tube, 2. Inflatable cuff, 9. lobar tube, 10. cuff of the lobar tube, 13. ventilator, 14. respiratory circuit inspiratory limb, 15. respiratory circuit expiratory limb, 17. trachea, 18. carina, 19. right upper lobe, 20. right upper lobe bronchi, 21. right middle lobe, 22. right middle lobe bronchi, 23. right lower lobe, 24. right lower lobe bronchi, 25. left upper lobe, 26. left upper lobe bronchi, 27. left lower lobe, 28. left lower lobe bronchi.

FIG. 6 illustrates the front view of the lungs and respiratory apparatus. 1. Endotracheal tube, 2. Inflatable cuff, 9. lobar tube, 10. cuff of the lobar tube, 13. ventilator, 14. respiratory circuit inspiratory limb, 15. respiratory circuit expiratory limb, 17. trachea, 18. carina, 19. right upper lobe, 20. right upper lobe bronchi, 21. right middle lobe, 22. right middle lobe bronchi, 23. right lower lobe, 24. right lower lobe bronchi, 25. left upper lobe, 26. left upper lobe bronchi, 27. left lower lobe, 28. left lower lobe bronchi, 32. therapeutic solution.

FIG. 7 illustrates the front view of the lungs. 17. trachea, 18. carina, 19. right upper lobe, 20. right upper lobe bronchi, 21. right middle lobe, 22. right middle lobe bronchi, 23. right lower lobe, 24. right lower lobe bronchi, 25. left upper lobe, 26. left upper lobe bronchi, 27. left lower lobe, 28. left lower lobe bronchi, 32. therapeutic solution delivered to the left lower lobe.

FIG. 8 illustrates the front view of the lungs and respiratory apparatus. 1. Endotracheal tube, 2. Inflatable cuff, 9. lobar tube, 10. cuff of the lobar tube, 13. ventilator, 14. respiratory circuit inspiratory limb, 15. respiratory circuit expiratory limb. 17. trachea, 18. carina, 19. right upper lobe, 20. right upper lobe bronchi, 21. right middle lobe, 22. right middle lobe bronchi, 23. right lower lobe, 24. right lower lobe bronchi, 25. left upper lobe, 26. left upper lobe bronchi, 27. left lower lobe, 28. left lower lobe bronchi, 32. therapeutic solution.

FIG. 9 illustrates the front view with magnification of intervention on left mainstem bronchus. 1. endotracheal tube, 2. tracheal cuff of endotracheal tube, 17. trachea, 18. carina, 25. left upper lobe, 26. left upper lobe bronchi, 27. left lower lobe, 28. left lower lobe bronchi, 30. flexible bronchoscope, 33. endoscopic forceps, 34. left mainstem bronchi, 35. endobronchial neoplasm or tumor, 36. cryoablation catheter.

FIG. 10 illustrates the perspective view of endotracheal device-1. endotracheal tube, 2. tracheal cuff, 4. tracheal tube connector, 5. pilot line for tracheal cuff, 6. connector for insertion of lobar tubes, 7. cap connector, 8. expandable sheath channel, 40. removable cover for the sheath channel, 41. rim for the attachment of removable cover, 45. opening for the sheath channel.

### DETAIL DESCRIPTION OF DRAWINGS

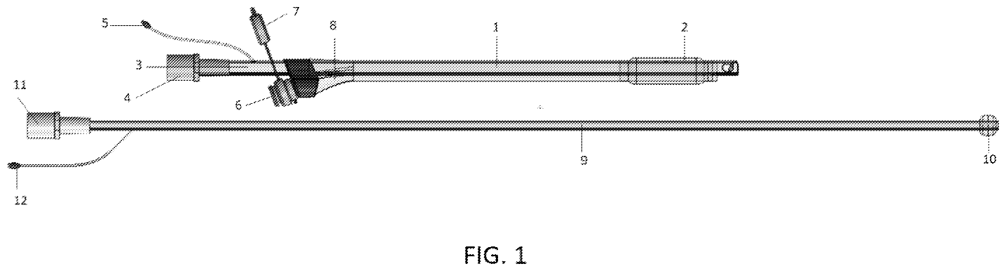

In FIG. 1, the endotracheal tube (1) and the lobar tube (9) are displayed separately, the tracheal tube on the top and the lobar tube on the bottom.

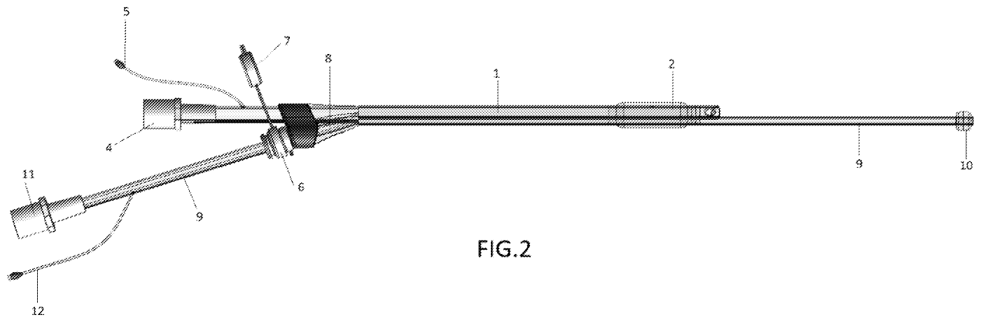

In FIG. 2, the lobar tube (9) is inserted through the sheath channel (8) on the tracheal tube 1.

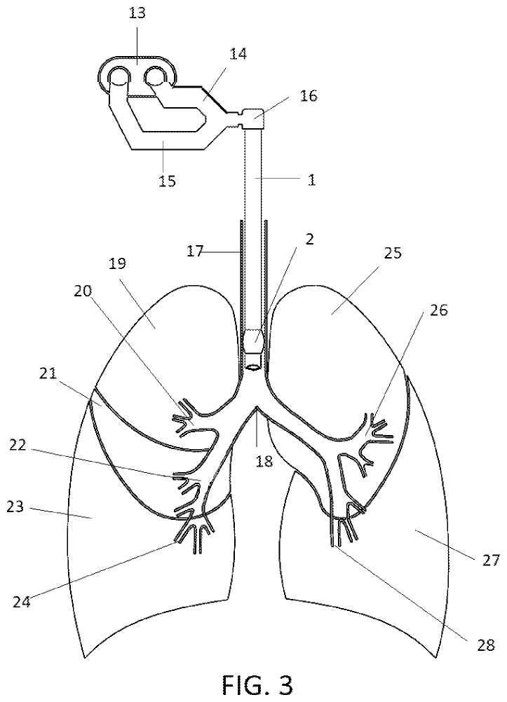

In FIG. 3, the endotracheal tube (1) is positioned in the trachea (17), above the carina (18). The ventilation of the lungs is provided by ventilator (13), respiratory circuit (14 & 15), and connector (16) with the endotracheal tube. The ventilation is distributed to all lung lobes.

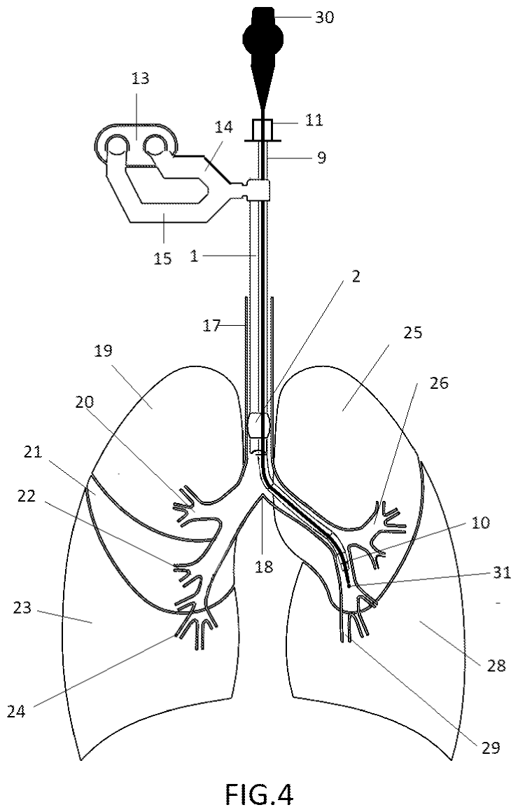

In FIG. 4, the lobar tube (9), placement is guided by flexible bronchoscope (30), by imaging the tracheal, bronchial tree with optic piece (31). The lobar tube (9), is placed over the bronchoscope (30), and they both are inserted through the sheath of the tracheal tube. The cuff (10), for the bronchial tube (9), is deflated during the placement.

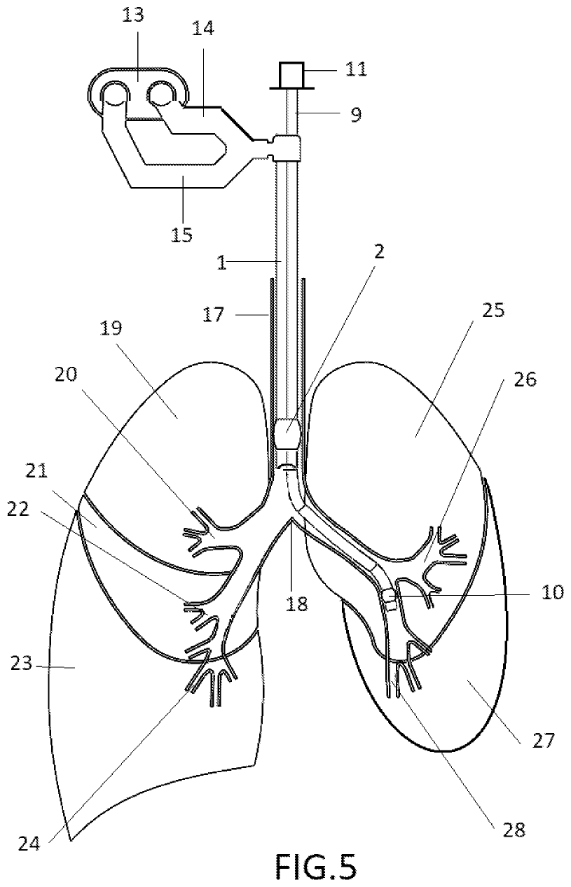

In FIG. 5, after the proper position of the lobar tube (9) in this example on the left lower lobe bronchi (28), the inflation of the cuff (10), and opening of the connector (11) to the atmosphere results in deflation or atelectasis of the lobe (27). The remaining lung lobes (19, 21, 23, and 25) and ventilator (13). The cuff (10) of the lobar tube allows selective isolation of the lobe for the administration of specific therapy and simultaneous ventilation of the rest of the lungs.

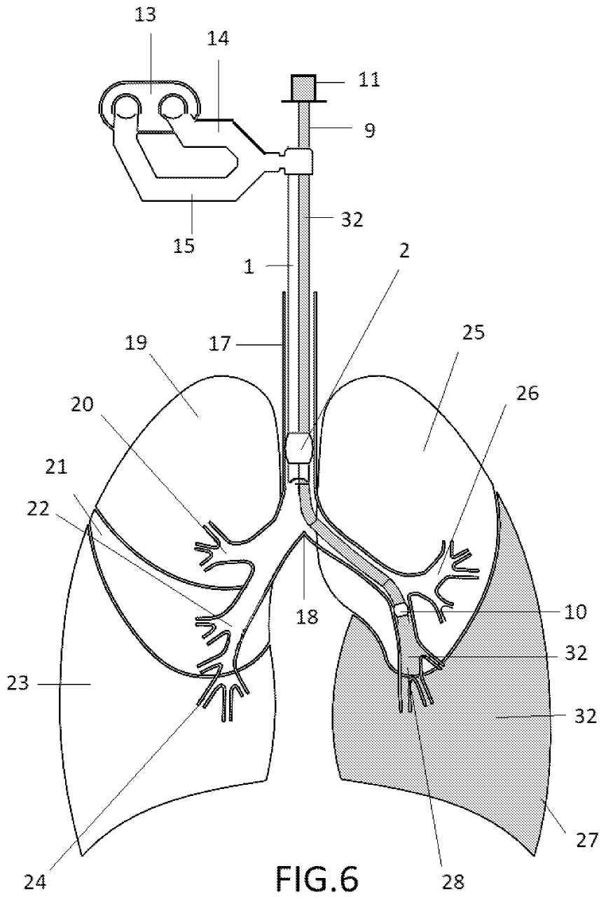

In FIG. 6, the therapeutic solution (32), is delivered through tube (9), into the left lower lobe bronchi (28) and left lower lobe (27). The remaining lobes (19, 21, 23 and 25) are simultaneously ventilated and connected to the tracheal tube (1). The inflated cuff 10 from the lobar tube (9), provides isolation of respective lobe (27), for delivery of a therapeutic solution and prevents the spilling or contamination of the remaining lobes (19, 21, 23, and 25 responsible for ventilation and oxygenation.)

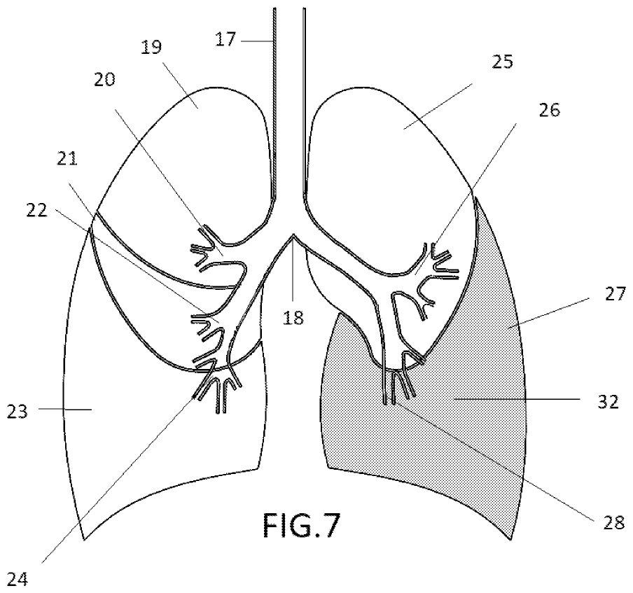

In FIG. 7, the lobar and tracheal tubes are removed after imaging confirmation that specific therapy (32) has been delivered to the left lower lobe (32).

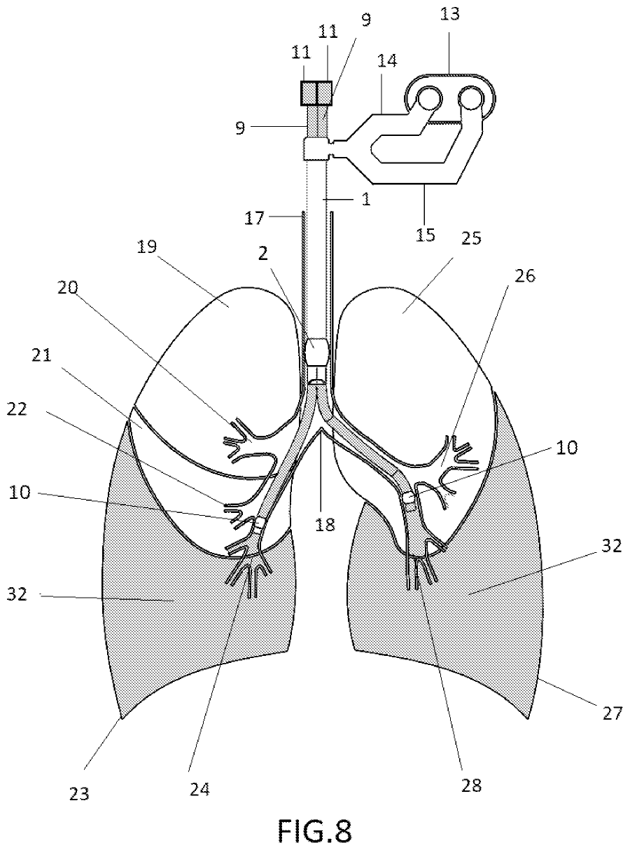

In FIG. 8, two lobar tubes (9), are placed on the right lower lobe (23 and left lower lobe (27), and with inflation of both cuffs (10), provides isolation of the respective lobes (23) and (27) for delivery of therapy (32), and simultaneous ventilation of lobes (19, 21, and 25).

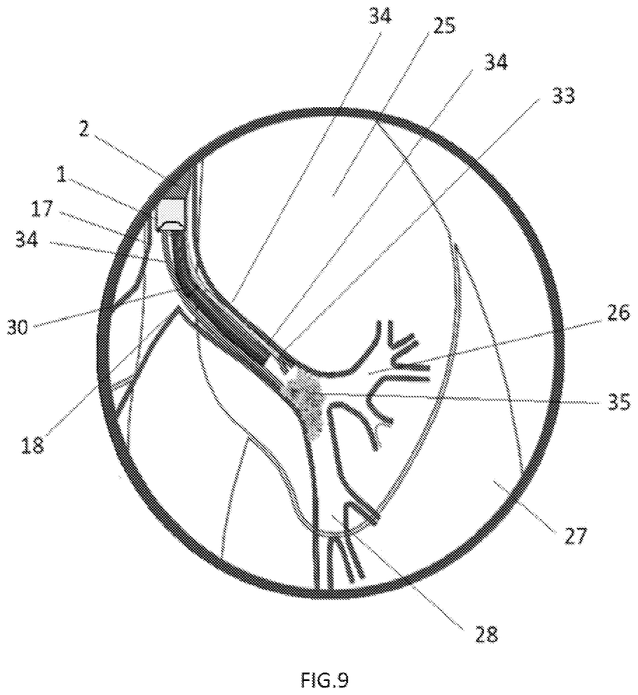

In FIG. 9), interventional bronchoscopy procedure with debulking of a tumor (35), showing a bronchoscope (30), cryoablation catheter (34), endoscopic forceps (33), passing through the channel posterior to the endotracheal tube, to perform the procedure. The tumor (35) is causing almost complete obstruction of the left main bronchi (34) bifurcation in left upper bronchi (26) and left lower bronchi (27). The obstruction prevents the proper ventilation of left upper (25) and left lower (27) lobe.

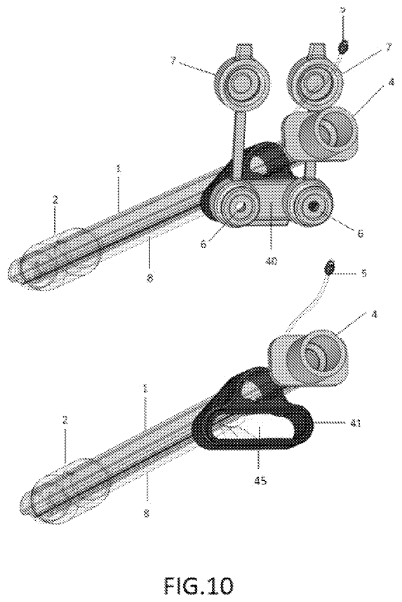

In FIG. 10, on top, the endotracheal tube (1), and connectors (6) to the channel (8), the channel allows placement of lobar tubes, and multiple other instruments for interventional bronchoscopy. The channel (8) on the sheath is independent from the channel of the endotracheal tube used for ventilation channel. If the channel is not in use the connector cap (7) should attached to the opening (6). On the bottom image, the cover for the connector (40) is open giving wide access to the channel (8) opening (45). This feature is designed for the removal of large foreign bodies, clots, secretions, tumor fragments. The cover for the connector (40), can be reattached to the rim (41), to continue necessary intervention.

The technique for lobar delivery of drugs and biologics consists of is selective placement of the lobar tube, lobe atelectasis, injection of the therapies in a liquid solution, followed by high liquid flow infusion to carry larger particles across all the bifurcations of the tracheal, bronchial tree, and reach the alveoli.

The therapeutic on interest is diluted in a liquid solution that is equal to the volume of the lobar tube and administered after the lobe atelectasis. After the administration, the therapeutic is flushed with positive pressure with saline solution with the same estimated volume of the lung lobe. The lobe volume is estimated based on computed tomography imaging or with a flowmeter during the deflation and atelectasis of the lobe.

The current approach to respiratory failure includes the initiation of mechanical ventilation and intravenous drugs that will hopefully reduce the disease progression and restore pulmonary function. Unfortunately, ventilator-induced lung injury is a frequent issue that can further deteriorate pulmonary function.

The delivery of large particles or cells into the lungs would likely not work by the inhalation route because cell mass and possible desiccation of the cells make them not viable. The delivery in liquid form in small volume will likely sediment in the tracheal, bronchial tree and not reach the alveoli either.

The indiscriminate infusion of large amounts of fluid into the lungs can potentially cause issues with ventilation and oxygenation due to surfactant dysfunction, flooding of multiple lobes but also not reaching areas of interest to deliver the therapies. The surface tension created by air and liquid would likely create bubbles, gas compression, and airlock phenomena, preventing the particles from reaching the alveoli.

The target lobe delivery through a small catheter of the channel of scope would likely not generate enough flow to bring the cells across the tracheobronchial bifurcations and reach the alveolar.

Here is described a new method of administration of drugs and cells to specific lung lobes.

We start with the positioning of a selective lobe device that allows isolation and atelectasis of one specific lobe and simultaneous ventilation of the rest of the lungs. After the lobe isolation, we measure the volume of the lobar tube and lung lobe. The cells we diluted to the same volume of the tube, followed by saline flush or infusion under positive pressure with the same volume of the tube.

The selective lobe delivery is an attractive option for the following reasons:
1—targeted and imaging-guided by computed tomography and bronchoscopy to a specific diseased lobe that is likely not contributing to global oxygenation.
2—safer—prior to the administration, the lobe can be isolated and not ventilated, and a drop in oxygenation can be assessed prior to the administration.
3—more effective—the lobe atelectasis prior to the infusion will remove all the air on the tissue, allowing diffusion on the parenchyma without air-liquid surface tension of backpressure that could prevent the liquid from reaching the alveoli.
4—allows for continuous and repeated infusion with the liquid in a controlled circumstance, and confirmation of delivery be done with imaging.
5—positive pressure infusion can be done simulating the respiratory cycle.

The invention herein consists of an airway device that allows for selective lobar isolation for the administration of drugs and biologics in liquid solution deeply into lung tissue, with simultaneous ventilation of the rest of the lungs. In addition, the novel airway device facilitates the performance of interventional bronchoscopy procedures such as biopsies, removal of foreign bodies, tumor debulking and placement of stents. The advantage of having separate ventilation and working channels allows continuous view and use of instruments without interruption necessary to remove structures from the airway.

1. A respiratory apparatus assembly comprising:
(a) an endotracheal tube device comprising elongated body forming a primary lumen, an expandable secondary channel or lumen formed by a plastic sheath that runs along the outside of the tube, a first connector, and a second connector; the elongated body having a proximal portion that remains external to a subject during use and a tracheal tube portion that is inserted into the trachea of a subject, the tracheal tube portion having a tracheal cuff around the tracheal portion of the tube that can be expanded to seal against the trachea; the secondary lumen is located outside the endotracheal tube and inside the tracheal cuff; the first connector is located external to the patient and on the proximal end of the endotracheal tube; and the second connector is positioned distal to the first connector and is located external to the patient, the second connector comprises a removable cover that when removed provides access to the secondary channel or lumen, wherein the cover contains two entry ports with silicon valves and caps, The valves prevent leak during instrumentation and the cap provides seal necessary for mechanical ventilation.
(b) a lobar tube or selective delivery device comprising an elongated body forming a lobar lumen, the lobar tube configured to be inserted into the lumen of the endotracheal tube device, the lobar tube having a distal inflatable cuff to isolate the lobe from the rest of the lungs and a proximal lobar tube connector; wherein the lobar tube is configured to be position in one lobe of the lung;
wherein the endotracheal tube and lobar tube forms an assembly with isolated lumens, (i) a lobar tube lumen for access through the lobar tube for delivery of therapies and (ii) an endotracheal tube lumen for access through the endotracheal tube for ventilating the lungs;
wherein the assembly is configured for lobe isolation and for the administration of therapies in a liquid format with simultaneous ventilation of the other lung lobes to maintain safe oxygenation.

2. The device of claim 1, configured to provide independent ventilation and working channels for interventional bronchoscopy, providing continuous visualization and instrumentation without interruption of the procedure to remove a scope.

3. The device of claim 1, further comprising an expandable sheath and a removable cap on the sheath compartment for removal of airway foreign bodies, clots, tumor or secretions; or for insertion of larger airway devices such as stents, coils, clips or multiple different instruments used in bronchoscopy.

4. A therapeutic delivery system for delivery of diagnostic or therapeutic agents into lung, lobes, or lung segments comprising a device of claim 1.

5. The delivery system of claim 4, wherein the therapeutic delivered is a liquid, aerosol, or gas.

6. The delivery system of claim 4, wherein the system is configured to be placed in lobar tubes by radiologic imaging assisted flexible bronchoscopy or robotic-assisted navigational bronchoscopy during use.

7. The delivery system of claim 4, wherein the system is configured to as a delivery for cell therapy or gene therapy.

8. A method for lobar delivery of the drugs and biologics in liquid solution comprising administering the liquid solution to a specific lobe using the device of claim 1, wherein dilution is based on tube volume and infusion flush is equal to volume of the lung lobe.

9. A respiratory apparatus assembly comprising:
(a) an endotracheal tube comprising:
(i) an elongated body forming a primary lumen, the elongated body having a proximal portion that remains external to a subject during use and a tracheal tube portion that is inserted into the trachea of a subject, the tracheal tube portion having a tracheal cuff around the tracheal portion of the tube that can be expanded to seal against the trachea;
(ii) an expandable sheath external to the endotracheal tube forming an expandable secondary lumen, the secondary lumen is positioned outside the endotracheal tube and inside the tracheal cuff;
(iii) a first connector located external to the patient and on the proximal end of the endotracheal tube;
(iv) a second connector positioned distal to the first connector and is located external to the patient, the second connector comprises a removable cover that when removed provides access to the secondary lumen, wherein the cover contains two entry ports with silicon valves and caps, the valves configured to prevent leak during instrumentation and the cap configured to provide a seal necessary for mechanical ventilation;
(b) a lobar tube or selective delivery device comprising:
(i) an elongated body forming a lobar lumen, the lobar tube configured to be inserted into the secondary lumen of the endotracheal tube, the lobar tube having a distal inflatable lobar cuff to isolate the lobe from the rest of the lungs, wherein the lobar tube is configured to be position in one lobe of the lung;
(ii) a proximal lobar tube connector;
wherein the endotracheal tube and lobar tube are configured to form an assembly with isolated lumens when in use, the lobar tube lumen configure to provide access through the lobar tube for delivery of therapies and an endotracheal tube lumen configured to provide access through the endotracheal tube for ventilating the lungs, the assembly providing lobe isolation for administration of therapies in a liquid format to one lobe with simultaneous ventilation of other lung lobes to maintain safe oxygenation when in use.

10. The assembly of claim 9, configured to provide independent ventilation and working channels for interventional bronchoscopy, providing continuous visualization and instrumentation without interruption of the procedure to remove a scope.

11. The assembly of claim 9, wherein the expandable sheath includes a removable proximal cap, the sheath lumen configured to provide for (i) removal of airway a foreign body, a clot, a tumor, or secretions, and/or (ii) insertion of larger airway devices such as stents, coils, clips or multiple different instruments used in bronchoscopy.

12. A therapeutic delivery system for delivery of diagnostic or therapeutic agents into lung, lobes, or lung segments comprising a device of claim 9 and one or more of a therapeutic delivery device and/or a ventilation device.

13. The delivery system of claim 12, wherein the therapeutic device is configured to deliver a liquid, aerosol, or gas.

14. The delivery system of claim 12, where the endotracheal tube and lobar tube can be used as an assembly or independently allowing conversion from a single-lumen tube to a double or triple-lumen tube depending on the number of lobar tubes placed through the expandable sheath.

15. The delivery system of claim 12, where the positioning of the lobar tube provides for delivery and/or two-lung ventilation, one-lung ventilation, or selective lobar access or lung segment access.

16. The assembly of claim 9, wherein the lobar tube further includes sensors capable of measuring physiological parameters such as pressure, flow, temperature, and volume of the isolated lung segment and the therapy administrated, to facilitate immediate adjustment of therapy based on the monitored parameters and targeted therapeutic interventions.

17. The assembly of claim 9, further comprising an integrated temperature control mechanism into the lobar tube or the endotracheal tube, the integrated temperature control mechanism configured to modulate the temperature of the therapeutic agents or gases being delivered.

18. A method of deploying the delivery system of claim 14, comprising using radiologic imaging-assisted flexible bronchoscopy or robotic-assisted navigational bronchoscopy to position the delivery system.

19. The method of claim 18, wherein the lobar tube is placed in the left or right main bronchi, lobar bronchi, or segmental bronchi.

20. The method of claim 18, further comprising inflating the lobar tube cuff isolating lung zones distal to the cuff to be used for selective delivery, ventilation, or recruitment.

21. The method of claim 18, further comprising delivering a cell therapy or gene therapy once the delivery system is in place.

22. A method for lobar delivery of the drugs and biologics in a liquid solution comprising isolating a lobe by inflating a lobar tube cuff and administering the liquid solution to an isolated lobe using the device of claim 9, wherein dilution is based on tube volume and infusion flush is equal to the volume of the lung lobe.

23. The method of claim 22, further comprising deflating, inflating, delivering therapeutics, ventilating, or recruiting lung zones distal to the engaged lobar cuff.

24. The method of claim 22, further comprising delivering a solution to lung tissue for lobar lavage in lobar pneumonia or alveolar proteinosis.

25. The method of claim 22, further comprising treating bronchopleural fistulas and collapsed lobes while the tracheal lumen channel ventilates the remaining lung zones.

26. The method of claim 22, further comprising delivering antibiotic drugs to treat multi-resistant germs.

27. The method of claim 22, further comprising delivering perfluorocarbon and/or surfactant to maintain lobe expansion.

28. The method of claim 22, further comprising delivering chemotherapy and/or radiotherapy to treat lung cancer.

29. The method of claim 22, further comprising isolating lobar delivery or recruitment while simultaneously ventilating remaining lung zones for lung graft optimization.

30. The method of claim 22, further comprising using selective delivery in combination with hyperbaric oxygen therapy, wherein the lobar tube is configured to deliver oxygen at a pressure higher than atmospheric pressure selectively to one lung or lobar regions, while the non-treated lung or lobes receive normal ventilation, enabling enhanced oxygen absorption and tissue repair without interrupting overall pulmonary function.
